(19) United States
(12) Patent Application Publication
(10) Pub. No.: US 2006/0002998 A1
(43) Pub. Date: Jan. 5, 2006

(54) Pharmaceutical dosage forms of biguanide-sulfonylurea combinations

(76) Inventors: Anupam Trehan, Delhi (IN); Sumit Madan, Delhi (IN); Vinod Kumar Arora, Delhi (IN); Rajiv Malik, Delhi (IN)

Correspondence Address: RANBAXY INC., 600 COLLEGE ROAD EAST, SUITE 2100, PRINCETON, NJ, 08540, US

(21) Appl. No.: 10/534,908

(22) PCT Filed: Nov. 17, 2003
(86) PCT No.: PCT/IB03/05206

§ 371 Date: May 13, 2005

(30) Foreign Application Priority Data
Nov. 15, 2002 (IN) 1154/DEL/2002

(51) Int. Cl. A61K 9/48; A61K 9/22; A61K 31/175; A61K 31/155

(52) U.S. Cl. 424/451; 424/468; 514/592; 514/635

(57) ABSTRACT

The present invention relates to orally administered pharmaceutical compositions that include a combination of antidiabetic agents wherein one agent is present in an extended release form and the other agent is present in an immediate release form. For example, in one embodiment the dosage form includes an extended release layer that includes a biguanide; and an immediate release layer that includes a sulfonylurea.

### FIELD OF THE INVENTION

The present invention relates to orally administered pharmaceutical compositions that include a combination of antidiabetic agents wherein one agent is present in an extended release form and the other agent is present in an immediate release form.

### BACKGROUND OF THE INVENTION

Diabetes mellitus is a term generally used to refer to various pathological states characterized by hyperglycemia and altered metabolism of lipids, carbohydrates and proteins. These conditions also are often associated with other co-morbidities, such as obesity and an increased risk of cardiovascular disease.

Diabetic conditions are generally classified as either insulin-dependent diabetes mellitus (IDDM, Type I diabetes) or non-insulin-dependent diabetes mellitus (NIDDM, Type II diabetes).

Virtually all forms of diabetes are due to a decrease in the circulating concentration of insulin (insulin deficiency) and/or a decrease in the response of peripheral tissues to insulin (insulin resistance). These abnormalities lead to alterations in the metabolism of carbohydrates, lipids, ketones and amino acids, and a hyperglycemic condition. IDDM appears to have an autoimmune etiology that results in destruction of B islet cells in the pancreas and leads to an inability to produce insulin. The etiology of NIDDM, the most prevalent form of diabetes, is more complex and possibly heterogeneous. NIDDM patients generally have a loss of B-cell volume, decreased circulating levels of insulin, and insulin resistance.

A variety of antidiabetic compounds are known. For example, sulfonylureas are a group of drugs that induce hypoglycemia by stimulating insulin release from the pancreas. Suitable sulfonyl ureas include acetohexamide, glibenclamide (glyburide), glipizide, gliclazide, glimepiride, tolazamide and tolbutamide. Prior to 1995, sulfonyl ureas were the most widely utilized antidiabetics for the treatment of NIDDM. These act by augmenting insulin secretion from the beta cells in the pancreas. Glyburide is available as tablets of 1.25 mg, 2.5 mg, and 5 mg strengths for oral administration and is administered twice a day. Glipizide tablets are available in 5 mg and 10 mg tablets. Similarly, glimperide is available in 1 mg, 2 mg, and 4 mg tablets and is administered once daily.

Biguanides are another group of drugs which were first introduced in the mid 1950's and have shown efficacy in the treatment of hyperglycemia. The best-known agents of this type include metformin, phenformin and buformin, with metformin being the most well known compound of this class. Metformin has been widely prescribed for lowering blood glucose in patients with NIDDM, is marketed as Glucophage tablets containing 500 mg, 850 mg, or 1000 mg of metformin hydrochloride and has a maximum recommended dose of 2550 mg per day. However, being a short acting drug, metformin requires twice-daily or three-times-a-day dosing (500-850 mg tab 2-3×/day or 1000 mg bid with meals). Unlike the sulfonylureas, biguanides do not induce release of insulin from the pancreas. It is thought that its effects are mediated by increasing insulin activity in peripheral tissues, reducing hepatic glucose output due to inhibition of gluconeogenesis, and reducing the absorption of glucose from the intestine.

These agents are often given in combination with drugs that increase the output of insulin from the pancreas, such as the sulfonylureas. This combination sometimes results in greater efficacy, the ability to use lower doses of the drugs, and reduced adverse side effect profile. Adverse events associated with the administration of biguanides include anorexia, nausea, vomiting and diarrhea. The adverse events may be partially avoided by either reducing the initial and/or maintenance dose using an extended-release dosage form. Another advantage of an extended-release dosage form is a reduction in the frequency of administration. Findings suggest that extended-release dosage form of a biguanide may improve the quality of therapy in patients with NIDDM.

Studies have shown that a combination of insulin secretion enhancers and insulin sensitivity enhancers has a remarkable effect on glycemic control. The different mechanisms of action in targeting hyperglycemia are complimentary and capable of providing a remedy for both the deficiency in insulin secretion and insulin sensitivity conditions. The combination therapy therefore plays an important therapeutic role because it provides an effective metabolic control in NIDDM patients in whom therapy with only sulfonylureas or only biguanides becomes ineffective with time.

The use of combinations of metformin (a biguanide) and glyburide (a sulfonylurea) has been demonstrated to be synergistic in clinical trials when compared with the use of the individual agents separately (see Physician's Desk Reference 2000, page 832). The monograph also discloses the use of combinations of metformin and sulfonylureas for patients not controlled on metformin alone. Several references pertain to pharmaceutical compositions having combinations of biguanides and sulfonylureas providing for controlled or immediate release of both of the drugs. For example, a unit-dose combination of metformin and glipizide as an immediate release formulation is commercially available (Zidmin™ tablets, Wockhardt), and a combination dosage form of metformin and glyburide for immediate release is described in U.S. Pat. No. 6,303,146 to Bonhomme et al.

Extended release tablets that employ either a biguanide alone or a sulfonylurea drug alone have been described in the prior art. For example, WO 96/08243 discloses a controlled release dosage form that contains only metformin hydrochloride as the active ingredient, and employs a hydrogel to push the active ingredient from the dosage form. Similarly, U.S. Pat. Nos. 5,545,413; 5,591,454; and 5,091,190 disclose controlled release dosage forms that contain only the drug glipizide, and employ a hydrogel to push the active ingredient from the dosage form.

U.S. Pat. Nos. 6,099,862 and 6,284,275, both to Chen et al., describe a combination composition for the simultaneous controlled release of a biguanide and a sulfonylurea. The composition comprises a core containing the two active agents along with other excipients and a semipermeable controlled release coating from which the release of the active agents is controlled by the presence of at least one passageway in the coat.

Although combinations of two antidiabetic agents are well known in the art and are convenient to formulate, a combination providing extended release of a water-soluble active, i.e., a biguanide, and immediate release of a water-insoluble or sparingly soluble active, i.e., sulfonylurea, is difficult to achieve using a simple and cost-effective process.

### SUMMARY OF THE INVENTION

In one general aspect there is provided a solid pharmaceutical dosage form for oral administration. The dosage form includes an extended release layer that includes a biguanide; and an immediate release layer that includes a sulfonylurea.

Embodiments of the dosage form may include one or more of the following features. For example, the biguanide may be one or more of metformin, phenformin, and buformin and, in particular, metformin. The sulfonylurea may be one or more of glipizide, glimepiride, glibornuride, glyburide, glisoxepide, gliclazide, acetohexamide, chlorpropamide, tolazamide and tolbutamide and, in particular, glimepiride.

After oral administration, the biguanide may be released over a period of about 4 hours to about 36 hours and, more particularly, over a period of about 8 to about 24 hours.

The dosage form may be tablets or capsules. The tablet may include a coating. The capsules may include one or more of pellets, beads, granules, multiparticulates, tablets and powder.

The extended release layer may be a matrix and the matrix may be a uniform mixture of the biguanide and one or more rate controlling polymers. The one or more rate-controlling polymers may be hydrophilic polymers, hydrophobic polymers, or a combination thereof. The matrix may further include one or more pharmaceutically acceptable excipients. The pharmaceutically acceptable excipients may be one or more of diluents, lubricants, disintegrants, binders, glidants, coloring agents, and flavoring agents.

The biguanide may be layered onto a pharmaceutically inert core or seed. The inert core or seed may be hydrosoluble or hydroinsoluble.

The immediate release outer layer may further include film-forming polymers and optionally other pharmaceutically acceptable excipients. The film-forming polymers may be water-soluble polymers. The pharmaceutically acceptable excipients may be one or more of plasticizers, opacifiers and colorants.

The dosage form may further include one or more of glitazones, insulin, alpha-glucosidase inhibitors, meglitinides, fibrates, statins, squalene synthesis inhibitors and angiotensin-converting enzyme inhibitors.

The dosage form may further include a wetting agent in the immediate release layer and the immediate release layer may include a sulfonylurea and the wetting agent in a weight ratio ranging from about 10:1 to about 1:25. The wetting agent may be one or more of hydrophilic and hydrophobic surfactants. The hydrophilic surfactant may be one or more of non-ionic surfactants, ionic surfactants or mixtures thereof. The hydrophobic surfactants may be one or more of alcohols; polyoxyethylene alkylethers; fatty acids; glycerol fatty acid monoesters; glycerol fatty acid diesters; acetylated glycerol fatty acid monoesters; acetylated glycerol fatty acid diesters, lower alcohol fatty acid esters; polyethylene glycol fatty acid esters; polyethylene glycol glycerol fatty acid esters; polypropylene glycol fatty acid esters; polyoxyethylene glycerides; lactic acid derivatives of monoglycerides; lactic acid derivatives of diglycerides; propylene glycol diglycerides; sorbitan fatty acid esters; polyoxyethylene sorbitan fatty acid esters; polyoxyethylene-polyoxypropylene block copolymers, polyethyleneglycols as esters or ethers, polyethoxylated castor oil; polyethoxylated hydrogenated castor oil, polyethoxylated fatty acid from castor oil or polyethoxylated fatty acid from castor oil or polyethoxylated fatty acid from hydrogenated castor oil.

The non-ionic surfactants may be one or more of alkylglucosides; alkylmaltosides; alkylthioglucosides; lauryl macrogolglycerides; caprylocaproyl macrogolglycerides, polyoxyethylene alkyl ethers; polyoxyethylene alkylphenols; polyethylene glycol fatty acid esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction products of polyols and at least one member of the group consisting of fatty acids, glycerides, vegetable oils, hydrogenated vegetable oils, and sterols; sugar esters, sugar ethers; sucroglycerides; and mixtures thereof.

The ionic surfactants may be one or more of alkyl ammonium salts; bile acids and salts, analogues, and derivatives thereof; fatty acid derivatives of amino acids, oligopeptides, and polypeptides; glyceride derivatives of amino acids, oligopeptides, and polypeptides; acyl lactylates; monoacetylated tartaric acid esters of monoglycerides, monoacetylated tartaric acid esters of diglycerides, diacetylated tartaric acid esters of monoglycerides, diacetylated tartaric acid esters of diglycerides; succinylated monoglycerides; citric acid esters of monoglycerides; citric acid esters of diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; and mixtures thereof.

The extended release layer may be a core and the immediate release layer covers at least a portion of the core. The dosage form may be a bilayered dosage form.

In another general aspect there is provided a process for preparing a solid, orally administered pharmaceutical dosage form of an extended release core of a biguanide and an immediate release layer of a sulfonylurea. The process includes (a) dispersing the biguanide in a solid matrix to form a core having a surface; and (b) layering the immediate release layer of the sulfonylurea on the surface of the core.

Embodiments of the process may include one or more of the following features. For example, layering the immediate release layer may further include layering one or more wetting agents. The sulfonylurea and the one or more wetting agents may be present in the immediate release layer in a weight ratio ranging from about 10:1 to about 1:25. The one or more wetting agents may be one or both of hydrophilic and hydrophobic surfactants. The hydrophilic surfactants may be one or more of non-ionic surfactants, ionic surfactants and mixtures thereof.

The hydrophobic surfactants may be one or more of alcohols; polyoxyethylene alkylethers; fatty acids; glycerol fatty acid monoesters; glycerol fatty acid diesters; acetylated glycerol fatty acid monoesters; acetylated glycerol fatty acid diesters, lower alcohol fatty acid esters; polyethylene glycol fatty acid esters; polyethylene glycol glycerol fatty acid esters; polypropylene glycol fatty acid esters; polyoxyethylene glycerides; lactic acid derivatives of monoglycerides; lactic acid derivatives of diglycerides; propylene glycol diglycerides; sorbitan fatty acid esters; polyoxyethylene sorbitan fatty acid esters; polyoxyethylene-polyoxypropylene block copolymers, polyethyleneglycols as esters or ethers, polyethoxylated castor oil; polyethoxylated hydrogenated castor oil, polyethoxylated fatty acid from castor oil or polyethoxylated fatty acid from castor oil or polyethoxylated fatty acid from hydrogenated castor oil.

The non-ionic surfactants may be one or more of alkylglucosides; alkylmaltosides; alkylthioglucosides; lauryl macrogolglycerides; caprylocaproyl macrogolglycerides, polyoxyethylene alkyl ethers; polyoxyethylene alkylphenols; polyethylene glycol fatty acid esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction products of polyols and at least one member of the group consisting of fatty acids, glycerides, vegetable oils, hydrogenated vegetable oils, and sterols; sugar esters, sugar ethers; sucroglycerides; and mixtures thereof.

The ionic surfactants may be one or more of alkyl ammonium salts; bile acids and salts, analogues, and derivatives thereof; fatty acid derivatives of amino acids, oligopeptides, and polypeptides; glyceride derivatives of amino acids, oligopeptides, and polypeptides; acyl lactylates; monoacetylated tartaric acid esters of monoglycerides, monoacetylated tartaric acid esters of diglycerides, diacetylated tartaric acid esters of monoglycerides, diacetylated tartaric acid esters of diglycerides; succinylated monoglycerides; citric acid esters of monoglycerides; citric acid esters of diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; and mixtures thereof.

The biguanide may be one or more of metformin, phenformin and buformin and, in particular, may be metformin. The sulfonylurea may be one or more of glipizide, glimepiride, glibornuride, glyburide, glisoxepide, gliclazide, acetohexamide, chlorpropamide, tolazamide and tolbutamide and, in particular, may be glimepiride.

After oral administration of the dosage form, the biguanide is released over a period of about 4 to about 36 hours and, in particular over a period of about 8 to about 24 hours.

The process may further include forming a tablet or a capsule and may still further include coating the tablet. The capsule may contain one or more of pellets, beads, granules, multiparticulates, tablets and powder.

The core may be a matrix. The matrix may further include a uniform mixture of the biguanide and one or more rate controlling polymers. The one or more rate-controlling polymers may be one or both of hydrophilic and hydrophobic polymers. The matrix may further include one or more pharmaceutically acceptable excipients. The pharmaceutically acceptable excipients may be one or more of diluents, lubricants, disintegrants, binders, glidants, colorants, and flavorants.

The biguanide may be layered onto pharmaceutically inert core or seeds. The inert core or seeds may be hydrosoluble or hydroinsoluble.

The immediate release layer may further include film-forming polymers and optionally other pharmaceutically acceptable excipients. The film-forming polymers may be water-soluble polymers. The pharmaceutically acceptable excipients may be one or more of plasticizers, opacifiers and colorants.

The process may further include placing a seal-coat over the core, wherein the seal-coat includes hydrophilic polymers.

In another general aspect there is provided a process for preparing a bilayered, solid, orally administered pharmaceutical dosage form of a biguanide and a sulfonylurea. The process includes (a) dispersing the biguanide in an extended release carrier base material; (b) separately dispersing the sulfonylurea in an immediate release carrier base material; and (c) compressing the materials of step a and step b to form the bilayered dosage form.

Embodiments of the process may include one or more of the following features. For example, the immediate release carrier base material may further include one or more wetting agents before or after dispersing the sulfonylurea. The sulfonylurea and the one or more wetting agents may be present in a weight ratio ranging from about 10:1 to about 1:25. The one or more wetting agents may be one or both of hydrophilic and hydrophobic surfactants. The hydrophilic surfactants may be one or more of non-ionic surfactants, ionic surfactants or mixtures thereof.

The hydrophobic surfactants may be one or more of alcohols; polyoxyethylene alkylethers; fatty acids; glycerol fatty acid monoesters; glycerol fatty acid diesters; acetylated glycerol fatty acid monoesters; acetylated glycerol fatty acid diesters, lower alcohol fatty acid esters; polyethylene glycol fatty acid esters; polyethylene glycol glycerol fatty acid esters; polypropylene glycol fatty acid esters; polyoxyethylene glycerides; lactic acid derivatives of monoglycerides; lactic acid derivatives of diglycerides; propylene glycol diglycerides; sorbitan fatty acid esters; polyoxyethylene sorbitan fatty acid esters; polyoxyethylene-polyoxypropylene block copolymers, polyethyleneglycols as esters or ethers, polyethoxylated castor oil; polyethoxylated hydrogenated castor oil, polyethoxylated fatty acid from castor oil or polyethoxylated fatty acid from castor oil or polyethoxylated fatty acid from hydrogenated castor oil.

The non-ionic surfactants may be one or more of alkylglucosides; alkylmaltosides; alkylthioglucosides; lauryl macrogolglycerides; caprylocaproyl macrogolglycerides, polyoxyethylene alkyl ethers; polyoxyethylene alkylphenols; polyethylene glycol fatty acid esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction products of polyols and at least one member of the group consisting of fatty acids, glycerides, vegetable oils, hydrogenated vegetable oils, and sterols; sugar esters, sugar ethers; sucroglycerides; and mixtures thereof.

The ionic surfactants may be one or more of alkyl ammonium salts; bile acids and salts, analogues, and derivatives thereof; fatty acid derivatives of amino acids, oligopeptides, and polypeptides; glyceride derivatives of amino acids, oligopeptides, and polypeptides; acyl lactylates; monoacetylated tartaric acid esters of monoglycerides, monoacetylated tartaric acid esters of diglycerides, diacetylated tartaric acid esters of monoglycerides, diacetylated tartaric acid esters of diglycerides; succinylated monoglycerides; citric acid esters of monoglycerides; citric acid esters of diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; and mixtures thereof.

The biguanide may be selected from one or more of metformin, phenformin and buformin and, in particular, may be metformin. The sulfonylurea may be selected from one or more of glipizide, glimepiride, glibornuride, glyburide, glisoxepide, gliclazide, acetohexamide, chlorpropamide, tolazamide and tolbutamide and, in particular, may be gllimepiride.

After oral administration of the dosage form, the biguanide may be released over a period of about 4 to about 36 hours and, in particular, over a period of about 8 to about 24 hours.

The process may further include forming a tablet or a capsule and may still further include coating the tablet. The capsule may contain one or more of pellets, beads, granules, multiparticulates, tablets and powder.

The biguanide layer may be a matrix and the matrix may include a uniform mixture of the biguanide and one or more rate controlling polymers. The one or more rate-controlling polymers may be either or both of hydrophilic and hydrophobic polymers. The matrix may further include one or more pharmaceutically acceptable excipients. The pharmaceutically acceptable excipients may be one or more of diluents, lubricants, disintegrants, binders, glidants, colorants, and flavorants.

The biguanide may be layered onto pharmaceutically inert core or seeds. The inert core or seeds may be hydrosoluble or hydroinsoluble.

The immediate release carrier base material may further include film-forming polymers and optionally other pharmaceutically acceptable excipients. The film-forming polymers may be water-soluble polymers. The pharmaceutically acceptable excipients may be one or more of plasticizers, opacifiers and colorants.

The process may further include providing a seal-coat of one or more hydrophilic polymers between the two layers.

In another general aspect there is provided a method of treating non-insulin dependent diabetes mellitus in a patient in need thereof. The method includes administering a solid, pharmaceutical dosage form of the combination of a biguanide and a sulfonylurea, wherein the dosage form provides extended-release of the biguanide and immediate release of the sulfonylurea.

Embodiments of the method may include one or more of the following features or any feature described above. For example, the biguanide may be one or more of metformin, phenformin, and buformin and, in particular, may be metformin. The sulfonylurea may be one or more of glipizide, glimepiride, glibornuride, glyburide, glisoxepide, gliclazide, acetohexamide, chlorpropamide, tolazamide and tolbutamide and, in particular, may be glimepiride.

After oral administration of the dosage form, the biguanide may be released over a period of about 4 to about 36 hours and, in particular, over a period of about 8 to about 24 hours.

The dosage form may tablets or capsules and the dosage form may further include one or more of glitazones, insulin, alpha-glucosidase inhibitors, meglitinides, fibrates, statins, squalene synthesis inhibitors and angiotensin-converting enzyme inhibitors.

The details of one or more embodiments of the inventions are set forth in the description below. Other features, objects and advantages of the inventions will be apparent from the description and claims.

### DETAILED DESCRIPTION OF THE INVENTION

Hydrophobic therapeutic agents, i.e., therapeutic compounds having poor solubility in aqueous solution, can be difficult to formulate in a dosage form that provides effective administration of the therapeutic agent to patients. A well-designed formulation must, at a minimum, be capable of presenting a therapeutically effective amount of the hydrophobic compound to the desired absorption site, in an absorbable form. Even this minimal functionality is difficult to achieve when delivery of the hydrophobic therapeutic agent requires interaction with aqueous physiological environments, such as gastric and intestinal fluids. Pharmaceutical compositions for delivery of such hydrophobic therapeutic agents must carry the hydrophobic compound through the aqueous environment, while maintaining the hydrophobic compound in an absorbable form, and avoiding the use of physiologically harmful solvents or excipients.

A similar problem is faced in formulating extended release dosage forms for highly soluble therapeutic agents. The high solubility of the therapeutic agent requires the incorporation of a high percentage of a rate-controlling polymer to achieve a desired release profile and prolonged effect. Further, it is difficult to control the initial burst of the drug from the formulation.

Therefore, there exists a need for pharmaceutical compositions for oral administration that include a combination of a hydrophobic, water-insoluble therapeutic agent, i.e., a sulfonylurea, in an immediate release form and a highly water-soluble therapeutic agent, i.e., a biguanide, in an extended-release form that has the characteristics of achieving an effect over twenty four hours after once daily administration.

The invention provides a dosage form containing both a sulfonylurea and a biguanide. The sulfonylurea is contained in an immediate-release form so that it is released substantially immediately upon ingestion (i.e., upon swallowing). Generally, at least 80% of the sulfonylurea is released from the dosage form within an hour after administration. The biguanide, by contrast, is released in a sustained fashion—at least about 75% of the drug contained in the dosage form is released over a period of four to thirty six hours, preferably about eight to twenty four hours. The term “about” as used above and elsewhere herein means plus or minus 10% for each of the numerical limits.

The pharmaceutical compositions of the present invention can be administered orally in the form of tablets, such as coated tablets or bilayered tablets, or in the form of capsules containing pellets, beads, granules, multiparticulates, tablets, or powder.

Biguanide, as used herein, is intended to include metformin, phenformin and buformin and their salts, solvates, hydrates, and polymorphs. In particular, the biguanide may be metformin. Different salts of metformin that can be used include hydrochloride, acetate, maleate, fumarate, succinate, and other salts. The daily effective dose of metformin may range from about 500 mg to about 2550 mg and, in particular, the dose may be a single dose of about 500 mg to about 1000 mg. The biguanide may be present in an amount from about 40% to about 75% by weight of the total composition.

The biguanide may be incorporated in an extended release carrier base by dispersing in a rate-controlling polymer matrix, as described in our pending application, which is published as WO 03/028704. Alternatively, the biguanide may be layered onto pharmaceutically acceptable inert cores or seeds in admixture with rate-controlling polymers or surrounded by rate-controlling polymers.

The term matrix, as used herein, refers to a uniform mixture of a biguanide, rate-controlling polymers and, optionally, other excipients. The rate-controlling polymers may be hydrophilic, hydrophobic or a combination thereof. The rate-controlling polymers are uniformly dispersed throughout the matrix to achieve uniform drug release. Hydrophilic polymers include cellulose derivatives such as hydroxypropylcellulose, hydroxypropyl methylcellulose, hydroxyethylcellulose, hydroxymethylcellulose, carboxymethylcellulose, methylcellulose, sodium carboxymethylcellulose or combinations thereof. The hydrophobic polymers may include one or more of poly (ethylene) oxide, ethyl cellulose, cellulose acetate, cellulose acetate butyrate, hydroxypropyl methylcellulose phthalate, poly (alkyl) methacrylate, and copolymers of acrylic or methacrylic acid esters, waxes, shellac and hydrogenated vegetable oils.

In addition to the active and rate-controlling polymers, the matrix may include other excipients that act in one or more capacities as diluents, binders, lubricants, glidants, colorants or flavoring agents. The matrix may be made by any pharmaceutically acceptable technique that achieves uniform blending, e.g., dry blending, wet granulation, compaction, and fluid bed granulation.

Suitable diluents include pharmaceutically acceptable inert fillers, such as one or more of microcrystalline cellulose, lactose, dibasic calcium phosphate, mannitol, starch, sorbitol, sucrose, dextrose, maltodextrin and mixtures thereof.

Suitable binders may include one or more of polyvinyl pyrrolidone, lactose, starches, gums, waxes, gelatin, polymers and mixtures thereof.

Suitable lubricants include one or more of colloidal silicon dioxide, talc, stearic acid, magnesium stearate, magnesium silicate, polyethylene, sodium benzoate, sodium lauryl sulphate, fumaric acid, zinc stearate, paraffin, and mixtures thereof.

Suitable glidants include, for example, one or more of talc and colloidal silicon dioxide.

The matrix formed can be compressed to form tablets. Alternatively, the matrix can be formulated as a plurality of discrete, or aggregated, particles, pellets, beads or granules.

The inert core or seeds may be hydro soluble, such as sucrose, lactose, maltodextrin and the like, or hydro insoluble, such as microcrystalline cellulose, partially pregelatinized starch, dicalcium phosphate and the like. The biguanide and rate-controlling polymer can be coated as a single layer or as separate layers onto these inert cores; granulated with the inert cores; or mixed with inert cores, extruded, and spheronized to form pellets.

The coating may be applied to the inert/active core using a conventional coating pan, a spray coater, a rotating perforated pan, or an automated system, such as centrifugal fluidized granulator, a fluidized bed process, or any other suitably automated coating equipment.

The extended-release core that contains biguanide may optionally be coated to seal the core. The coated active cores may be dried under conditions effective for drying, e.g., in an oven or by means of gas in a fluidized bed.

Finally, these beads/pellets may be filled into capsules or compressed to form the tablets. The capsule dosage form may include a plurality of pellets, granules, or beads, or a single, compressed tablet that release the biguanide over an extended period of time.

The sulfonylurea as used herein is intended to include, but is not limited to, glipizide, glimepiride, glibornuride, glyburide, glisoxepide, gliclazide, acetohexamide, chlorpropamide, tolazamide, tolbutamide, and others, and other medicinally active and pharmaceutically acceptable forms from the sulfonylurea class of compounds, including their salts, solvates, hydrates, polymorphs, complexes and other such products. For example, suitable sulfonylureas for use in the present invention are described in U.S. Pat. Nos. 5,674,900 and 4,708,868, both of which are incorporated herein by reference in their entirety. In particular, the sulfonylurea used may be glimepiride. The daily effective dose of glimepiride may range from 1 mg to 10 mg and, in particular, the dose may be a single dose of about 2 mg to about 4 mg. The sulfonylurea may be present in an amount from about 0.05% to about 10% by weight of the total composition.

A sulfonylurea can be incorporated into the dosage form as an immediate release component in a variety of ways. For example, it can be incorporated into an exterior coating for a tablet from which it releases substantially immediately upon ingestion. Such a coating can similarly be applied to each of the particles that form a multiparticulate dosage form of, for example, granules or beads. If the dosage form is to be a capsule, the sulfonylurea can be contained in a single pellet inside the capsule from which it releases substantially immediately once the capsule shell dissolves. Alternatively, the sulfonylurea can be contained in several smaller pellets, be present as immediate release particles, or be present as an immediate release layer over the extended release cores or beads. Any conventional method may be used to prepare the layer of sulfonylurea. Conventional pharmaceutically acceptable excipients may be incorporated into this layer. These excipients may include one or more of diluents, binders, and lubricants.

The sulfonylurea coating composition may include one or more water-soluble polymers, such as polyvinyl pyrrolidine, hydroxypropyl cellulose, polyvinyl alcohol, hydroxypropyl methylcellulose and the like. The polymer may be applied as a solution in an organic solvent or as an aqueous dispersion. The solvent may be one or more of water; alcohols, such as ethyl alcohols or isopropyl alcohol; ketones, such as acetone or ethylmethyl ketone; and chlorinated hydrocarbons, such as dichloroethane and trichloroethane. The coating composition also may include one or more of plasticizers, opacifiers, and colorants. Any conventional coating equipment may be employed to facilitate coating, including a centrifugal fluidized bed coating apparatus, a pan coating apparatus, or a fluidized bed granulating coating apparatus.

Due to poor dispersibility in solvents, the film-coating composition that includes the sulfonylurea includes a wetting agent. Suitable wetting agents include hydrophilic and hydrophobic surfactants. Hydrophilic surfactants may include one or more of hydrophilic non-ionic surfactants, hydrophilic ionic surfactants, and combinations thereof.

Non-ionic surfactants may be selected from one or more of alkylglucosides; alkylmaltosides; alkylthioglucosides; lauryl macrogolglycerides; caprylocaproyl macrogolglycerides, polyoxyethylene alkyl ethers; polyoxyethylene alkylphenols; polyethylene glycol fatty acid esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction products of polyols and at least one member of the group consisting of fatty acids, glycerides, vegetable oils, hydrogenated vegetable oils, and sterols; sugar esters, sugar ethers; sucroglycerides; and mixtures thereof.

Ionic surfactants may be selected from one or more of alkyl ammonium salts; bile acids and salts, analogues, and derivatives thereof; fatty acid derivatives of amino acids, oligopeptides, and polypeptides; glyceride derivatives of amino acids, oligopeptides, and polypeptides; acyl lactylates; monoacetylated tartaric acid esters of monoglycerides, monoacetylated tartaric acid esters of diglycerides, diacetylated tartaric acid esters of monoglycerides, diacetylated tartaric acid esters of diglycerides; succinylated monoglycerides; citric acid esters of monoglycerides; citric acid esters of diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; and mixtures thereof.

Hydrophobic surfactants may be selected from one or more of alcohols; polyoxyethylene alkylethers; fatty acids; glycerol fatty acid monoesters; glycerol fatty acid diesters; acetylated glycerol fatty acid monoesters; acetylated glycerol fatty acid diesters, lower alcohol fatty acid esters; polyethylene glycol fatty acid esters; polyethylene glycol glycerol fatty acid esters; polypropylene glycol fatty acid esters; polyoxyethylene glycerides; lactic acid derivatives of monoglycerides; lactic acid derivatives of diglycerides; propylene glycol diglycerides; sorbitan fatty acid esters; polyoxyethylene sorbitan fatty acid esters; polyoxyethylene-polyoxypropylene block copolymers, polyethyleneglycols as esters or ethers, polyethoxylated castor oil; polyethoxylated hydrogenated castor oil, polyethoxylated fatty acid from castor oil or polyethoxylated fatty acid from castor oil or polyethoxylated fatty acid from hydrogenated castor oil.

The sulfonylurea and the one or more wetting agents are present in the pharmaceutical composition in a weight ratio ranging from about 10:1 to about 1:25.

One embodiment is a bilayered dosage form that includes the combination of a biguanide and a sulfonylurea. The term ‘bilayered’ as used herein encompasses solid dosage forms in which there are two separate drug layers with only one surface in contact with each other. These may be prepared by compressing additional granulation on a previously compressed granulation or alternatively by feeding previously compressed tablets into a machine and compressing another granulation layer around the preformed tablets.

Another embodiment includes providing a seal coat of hydrophilic polymers between the extended-release and immediate-release layers.

Other embodiments include additional or alternative modifications that involve coating the tablet with the polymer in order to modify the release of the drug. The solid dosage forms may be optionally coated with non-functional coatings well known in the art, or with coatings that further modify the release of the drug from the said dosage form. All such modifications as may be done and understood by those who are skilled in the art are within the scope of the present invention. For example, one such modification includes making the compositions into a layered tablet such that the composition provides extended release of more than one therapeutic agent, or extended release of one of the therapeutic agents and immediate or delayed release of the other therapeutic agent(s).

### EXAMPLE 1

| | INGREDIENTS | Mg/tablet |
|---|---|---|
| CORE | Metformin hydrochloride | 500 |
| | Microcrystalline cellulose | 245 |
| | Sodium carboxymethyl cellulose | 150 |
| | Purified water | q.s. |
| | Hydroxypropyl methylcellulose | 100 |
| | Magnesium stearate | 5 |
| SEAL | Hydroxypropyl methylcellulose E5 | 15.6 |
| COAT | Polyethylene glycol 4000 | 4.8 |
| | Titanium dioxide | 2.4 |
| | Talc | 1.2 |
| | Purified water | q.s. |
| ACTIVE | Glimepiride (20% extra to compensate for losses) | 1.2 |
| COAT | Caprylocaporyl macrogolglycerides | 14.4 |
| | Hydroxypropyl methylcellulose E5 | 29.35 |
| | Polyethylene glycol 4000 | 8.6 |
| | Titanium dioxide | 4.3 |
| | Talc | 2.15 |
| | Purified water | q.s. |

Procedure:
1. Metformin hydrochloride was milled through a 1 mm screen and mixed with microcrystalline cellulose and sodium carboxymethyl cellulose. The blend was sieved through a No. 44 mesh, transferred to a rapid mixer granulator, and wet granulated with purified water. The granules were dried in fluid bed dryer, sized through a multimill, and sifted through a No. 30 mesh.
2. Hydroxypropyl methylcellulose was separately sifted through a No. 30 mesh and mixed with granules in a low shear mixer. The blend then was mixed with magnesium stearate and compressed into tablets.
3. A coating dispersion was prepared by dispersing all of the ingredients of the seal coat in water. The tablets then were coated with this dispersion until obtaining a weight build up of 5%.
4. To prepare the active coat, caprylocaproyl monoglyceride was dissolved in purified water. To this solution, glimepiride was added with stirring to form a dispersion. The other ingredients of the active coat were added with stirring to this dispersion and the resulting dispersion then was spray-coated upon the tablets obtained from step 3 until obtaining a weight build up of 10%.

### EXAMPLE 2

| | INGREDIENTS | Mg/tablet |
|---|---|---|
| CORE | Metformin hydrochloride | 500 |
| | Microcrystalline cellulose | 245 |
| | Sodium carboxymethyl cellulose | 150 |
| | Hydroxypropyl methylcellulose | 100 |
| | Magnesium stearate | 5 |
| SEAL | Hydroxypropyl methylcellulose E5 | 15.6 |
| COAT | Polyethylene glycol 4000 | 4.8 |
| | Titanium dioxide | 2.4 |
| | Talc | 1.2 |
| | Purified water | q.s. |
| ACTIVE | Glimepiride equivalent to 2 mg (20% extra | 2.4 |
| COAT | to compensate for losses) | |
| | Caprylocaporyl macrogolglycerides | 14.4 |
| | Hydroxypropyl methylcellulose E5 | 28.15 |
| | Polyethylene glycol 4000 | 8.6 |
| | Titanium dioxide | 4.3 |
| | Talc | 2.15 |
| | Purified water | q.s. |

Procedure:
1. Metformin hydrochloride was milled through a 1 mm screen and mixed with microcrystalline cellulose and sodium carboxymethyl cellulose. The blend was sieved through a No. 44 mesh.
2. Hydroxypropyl methylcellulose was separately sifted through a No. 30 mesh and mixed with the blend of step 1 in a low shear mixer. The blend then was mixed with magnesium stearate, passed through roller compactor, and then milled again to form granules. These granules then were compressed into tablets.
3. A coating dispersion was prepared by dispersing all of the ingredients of the seal coat in water. The tablets then were coated with this dispersion until obtaining a weight build up of 5%.
4. To prepare the active coat, caprylocaproyl monoglyceride was dissolved in purified water. To this solution, glimepiride was added with stirring to form a dispersion. The other ingredients of the active coat were added with stirring to this dispersion. The resulting dispersion then was spray-coated onto the tablets obtained from step 3 until obtaining a weight build up of 8.0%.

A comparative dissolution profile was obtained of metformin hydrochloride in the innovator's marketed tablets (Glucophage XR 500 mg) and tablet formulation of Example 2. The dissolution was carried out in a USP Apparatus Type I (basket) at a speed of 100 rpm. The medium was 900 ml phosphate buffer at pH 6.8. The data obtained is provided in Table 1.

TABLE 1
Comparative dissolution profile of metformin hydrochloride
in Glucophage XR 500 mg vs tablets of Example 2

Percent (%) metformin
hydrochloride released

| Time (hrs) | Glucophage XR | Tablets (Example 2) |
|---|---|---|
| 0 | 0 | 0 |
| 1 | 29 | 28 |
| 4 | 60 | 64 |
| 8 | 83 | 91 |
| 12 | 99 | 100 |

From the results of Table 1, it is evident that almost all of the drug is released in twelve hours in both the formulations, thereby showing substantially similar dissolution profiles.

A comparative dissolution profile was obtained of glimepiride in the innovator's marketed tablets (Amaryl 2 mg) and the tablet formulation of Example 2. The dissolution was carried out in a USP Apparatus Type I at a speed of 100 rpm. The medium was 900 ml phosphate buffer at pH 8. The data obtained is provided in Table 2.

TABLE 2
Comparative dissolution profile of glimepiride in
Amaryl 2 mg vs tablets of Example 2

Percent (%) Glimepiride released

| Time (hrs) | Amaryl 2 mg | Tablets (Example 2) |
|---|---|---|
| 0 | 0 | 0 |
| 15 | 95 | 92 |
| 30 | 98 | 101 |
| 45 | 98 | 105 |

From the results of Table 2, it is evident that more than 90% of the drug is released in fifteen minutes in both the formulations, thereby showing substantially similar dissolution profiles.

### EXAMPLE 3

| | INGREDIENTS | mg/tablet |
|---|---|---|
| CORE | Metformin hydrochloride | 500 |
| | Microcrystalline cellulose | 245 |
| | Sodium carboxymethyl cellulose | 150 |
| | Hydroxypropyl methylcellulose | 100 |
| | Magnesium stearate | 5 |
| SEAL | Hydroxypropyl methylcellulose E5 | 15.6 |
| COAT | Polyethylene glycol 4000 | 4.8 |
| | Titanium dioxide | 2.4 |
| | Talc | 1.2 |
| | Purified water | q.s. |
| ACTIVE | Glimepiride equivalent to 2 mg (20% extra to | 2.4 |
| COAT | compensate for losses) | |
| | Hydroxypropyl methylcellulose E5 | 37.2 |
| | Polyethylene glycol 400 | 7.2 |
| | Titanium dioxide | 6.2 |
| | Talc | 12.0 |
| | Methylene chloride | q.s. |
| | Isopropyl alcohol | q.s. |

Procedure:
1. Metformin hydrochloride was milled through a 1 mm screen and mixed with microcrystalline cellulose and sodium carboxymethyl cellulose. The blend was sieved through a No. 44 mesh.
2. Hydroxypropyl methylcellulose was separately sifted through a No. 30 mesh and mixed with the blend in a low shear mixer. The blend then was mixed with magnesium stearate and compressed into tablets.
3. A coating dispersion was prepared by dispersing all of the ingredients of the seal coat in water. The tablets were coated with this dispersion until obtaining a weight build up of 2%.
4. To prepare the active coat, glimepiride was dissolved in a methylene chloride:isopropyl alcohol mix (2:1). The other ingredients of the active coat were added with stirring to this solution. The resulting dispersion then was spray-coated upon the tablets obtained from step 3 until obtaining a weight build up of 10%.

While several particular forms of the inventions have been illustrated and described, it will be apparent that various modifications and combinations of the inventions detailed in the text can be made without departing from the spirit and scope of the inventions. For example, a bilayered tablet that include extended-release biguanide in one layer and immediate-release sulfonylurea in another layer may be prepared according to the example given below.

### EXAMPLE 4

Preparation of Bilayered Tablets:

| | INGREDIENTS | Mg/tablet |
|---|---|---|
| Metformin | Metformin hydrochloride | 500 |
| layer | Microcrystalline cellulose | 245 |
| | Sodium carboxymethyl cellulose | 150 |
| | Hydroxypropyl methylcellulose | 100 |
| | Magnesium stearate | 5 |
| Seal Coat | Hydroxypropyl methylcellulose E5 | 15.6 |
| | Polyethylene glycol 4000 | 4.8 |
| | Titanium dioxide | 2.4 |
| | Talc | 1.2 |
| Piosulfonylurea | Glimepiride equivalent to 2 mg | 2.4 |
| layer | Lactose | 143 |
| | Microcrystalline cellulose | 20 |
| | Sodium starch glycolate | 6.0 |
| | Polyvinylpyrrolidone | 2.5 |
| | Magnesium stearate | 1.0 |
| | Purified water | q.s. |

Procedure:
1. Metformin hydrochloride was milled and mixed with microcrystalline cellulose and sodium carboxymethyl cellulose. The blend was sieved.
2. Hydroxypropyl methylcellulose was separately sifted and mixed with the blend of step 1 in a low shear mixer. The blend then was mixed with magnesium stearate and passed through a roller compactor and then milled again to form granules.
3. Glimepiride, lactose, microcrystalline cellulose and sodium starch glycolate were blended and granulated with a solution of polyvinylpyrrolidone in purified water.
4. The wet mass of step 3 was granulated, dried, and sifted.
5. The lubricated granules of metformin and glimepiride were compressed into bilayer tablets using a rotary compression machine.

Further, it is contemplated that any single feature or any combination of optional features of the inventive variations described herein may be specifically excluded from the claimed invention and be so described as a negative limitation. Accordingly, it is not intended that the invention be limited, except as by the appended claims.

1. A solid pharmaceutical dosage form for oral administration, the dosage form comprising:
an extended release layer comprising a biguanide; and
an immediate release layer comprising a sulfonylurea.

2. The dosage form of claim 1, wherein the biguanide comprises one or more of metformin, phenformin, and buformin.

3. The dosage form of claim 1, wherein the biguanide is metformin.

4. The dosage form of claim 1, wherein the sulfonylurea comprises one or more of glipizide, glimepiride, glibornuride, glyburide, glisoxepide, gliclazide, acetohexamide, chlorpropamide, tolazamide and tolbutamide.

5. The dosage form of clam 1, wherein the sulfonylurea is glimepiride.

6. The dosage form of claim 1, wherein after oral administration the biguanide is released over a period of about 4 to about 36 hours.

7. The dosage form of claim 6, wherein the biguanide is released over a period of about 8 to about 24 hours.

8. (canceled)

9. (canceled)

10. (canceled)

11. The dosage form of claim 1, wherein the extended release layer comprises a matrix.

12. The dosage form of claim 11, wherein the matrix comprises a uniform mixture of the biguanide and one or more rate controlling polymers.

13. (canceled)

14. The dosage form of claim 11, wherein the matrix further comprises one or more pharmaceutically acceptable excipients.

15. The dosage form of claim 14, wherein the pharmaceutically acceptable excipients comprise one or more of diluents, lubricants, disintegrants, binders, glidants, coloring agents, and flavoring agents.

16. The dosage form of claim 1, wherein the biguanide is layered onto a pharmaceutically inert core or seed.

17. The dosage form of claim 16, wherein the inert core or seed is hydrosoluble or hydroinsoluble.

18. The dosage form of claim 1, wherein the immediate release outer layer further comprises film-forming polymers and optionally other pharmaceutically acceptable excipients.

19. The dosage form of claim 18, wherein the film-forming polymers are water-soluble polymers.

20. The dosage form of claim 18, wherein the pharmaceutically acceptable excipients comprise one or more of plasticizers, opacifiers and colorants.

21. The dosage form of claim 1, further comprising one or more of glitazones, insulin, alpha-glucosidase inhibitors, meglitinides, fibrates, statins, squalene synthesis inhibitors and angiotensin-converting enzyme inhibitors.

22. The dosage form of claim 1, further comprising a wetting agent in the immediate release layer, wherein the immediate release layer comprises a sulfonylurea and the wetting agent in a weight ratio ranging from about 10:1 to about 1:25.

23. The dosage form of claim 22, wherein the wetting agent comprises one or more of hydrophilic and hydrophobic surfactants.

24. The dosage form of claim 23, wherein the hydrophilic surfactants comprises one or more of non-ionic surfactants, ionic surfactants or mixtures thereof.

25. (canceled)

26. (canceled)

27. (canceled)

28. The dosage form of claim 1, wherein the extended release layer comprises a core and the immediate release layer covers at least a portion of the core.

29. The dosage form of claim 1, wherein the dosage form comprises a bilayered dosage form.

30. A process for preparing a solid, orally administered pharmaceutical dosage form of an extended release core of a biguanide and an immediate release layer of a sulfonylurea, the process comprising:
a. dispersing the biguanide in a solid matrix to form a core having a surface; and
b. layering the immediate release layer of the sulfonylurea on the surface of the core.

31. The process of claim 30, wherein layering the immediate release layer further comprises layering one or more wetting agents.

32. The process of claim 31, wherein the sulfonylurea and the one or more wetting agents are present in the immediate release layer in a weight ratio ranging from about 10:1 to about 1:25.

33. (canceled)

34. (canceled)

35. (canceled)

36. (canceled)

37. (canceled)

38. (canceled)

39. (canceled)

40. (canceled)

41. (canceled)

42. The process of claim 30, wherein after oral administration the biguanide is released over a period of about 4 to about 36 hours.

43. The process of claim 42, wherein the biguanide is released over a period of about 8 to about 24 hours.

44. (canceled)

45. (canceled)

46. (canceled)

47. (canceled)

48. (canceled)

49. (canceled)

50. (canceled)

51. (canceled)

52. The process of claim 30, wherein the biguanide is layered onto pharmaceutically inert core or seeds.

53. (canceled)

54. The process of claim 30, wherein the immediate release outer layer further comprises film-forming polymers and optionally other pharmaceutically acceptable excipients.

55. (canceled)

56. (canceled)

57. The process of claim 30, further comprising placing a seal-coat over the core, wherein the seal-coat comprises hydrophilic polymers.

58. A process for preparing a bilayered, solid, orally administered pharmaceutical dosage form of a biguanide and a sulfonylurea, the process comprising:
a. dispersing the biguanide in an extended release carrier base material;
b. separately dispersing the sulfonylurea in an immediate release carrier base material; and
c. compressing the materials of step a and step b to form the bilayered dosage form.

59. The process of claim 58, wherein the immediate release carrier base material further comprises one or more wetting agents before or after dispersing the sulfonylurea.

60. The process of claim 59, wherein the sulfonylurea and the one or more wetting agents are present in a weight ratio ranging from about 10:1 to about 1:25.

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. The process of claim 58, wherein the biguanide is layered onto pharmaceutically inert core or seeds.

81. The process of claim 80, wherein the inert core or seeds are hydrosoluble or hydroinsoluble.

82. (canceled)

83. (canceled)

84. (canceled)

85. (canceled)

86. A method of treating non-insulin dependent diabetes mellitus in a patient in need thereof, the method comprising administering a solid, pharmaceutical dosage form of the combination of a biguanide and a sulfonylurea, wherein the dosage form provides extended-release of the biguanide and immediate release of the sulfonylurea.

87. The method of claim 86, wherein the biguanide comprises one or more of metformin, phenformin, and buformin.

88. The method of claim 86, wherein the biguanide is metformin.

89. The method of claim 86, wherein the sulfonylurea comprises one or more of glipizide, glimepiride, glibornuride, glyburide, glisoxepide, gliclazide, acetohexamide, chlorpropamide, tolazamide and tolbutamide.

90. The method of clam 86, wherein the sulfonylurea is glimepiride.

91. (canceled)

92. (canceled)

93. (canceled)

94. The method of claim 86, wherein the dosage form further comprises one or more of glitazones, insulin, alpha-glucosidase inhibitors, meglitinides, fibrates, statins, squalene synthesis inhibitors and angiotensin-converting enzyme inhibitors.
